(19) United States
(12) Patent Application Publication
(10) Pub. No.: US 2010/0227406 A1
(43) Pub. Date: Sep. 9, 2010

(54) METHOD FOR PURIFYING CELLS, RECOVERING CELLS, AND TRANSFECTING CELLS GENTLY

(75) Inventors: Ioanna Andreou, Hilden (DE); Ralf Himmelreich, Hilden (DE); Andrea Janosch, Hilden (DE)

Correspondence Address: Fanelli Strain & Haag PLLC, 1455 Pennsylvania Ave., N.W., suite 400, Washington, DC, 20004, US

(73) Assignee: QIAGEN GMBH

(21) Appl. No.: 12/601,810

(22) PCT Filed: May 21, 2008
(86) PCT No.: PCT/EP2008/056277

§ 371 Date: May 24, 2010

(30) Foreign Application Priority Data
May 25, 2007 (EP) 07010424.5

(51) Int. Cl. C12N 15/00; C12N 1/08; C12N 11/00; C12M 1/00

(52) U.S. Cl. 435/440; 435/270; 435/174; 435/283.1

(57) ABSTRACT

The present invention relates to a process for cell purification, for cell recovery from cultures and for the transfection of the cells under especially gentle conditions, where this process can additionally be coupled with subsequent isolation and purification of polynucleotides from the optionally transfected cells, and a kit and a device for the implementation of this process.

The present invention relates to a process for cell purification, for cell recovery from cultures and/or for the transfection of cells under especially gentle conditions, where this process can additionally be coupled with subsequent isolation and purification of polynucleotides from the optionally transfected cells, and a kit and a device for the implementation of this process.

Previous processes for cell recovery or purification of cells normally include a centrifugation step, whereby the cells are separated from a suspending medium by centrifugal force. This centrifugation step must be performed under conditions which have no adverse effect on the cells. For example, too high a centrifugal force leads to the destruction of the cells. Moreover, in this case not all the cells are taken, many remain in suspension. Since during centrifugation the cells must be removed from the sterile workbench several times, they are in addition exposed to the risk of becoming contaminated and this is a very critical point in cell experiments.

A further possible means of removing cells from liquid media is filtration, but here also conditions must be maintained which do not result in destruction of the cells. In addition, the cell loss in filtration devices is relatively high, since the cells can only be removed from the filter materials with difficulty afterwards.

A process which is also commonly used, in particular for selective, specific cell purification or recovery is the binding of cells to solid surfaces via specific molecules which “recognise” surface structures or surface proteins of these specific cells, for example binding via specific receptors or surface proteins of the cells.

The use of magnetic spherules, so-called “beads” for the purification of poly-nucleotides from liquid media has long been known. For example, EP-A 515 484, EP-A 764 206 and WO 01/71732 each describe a process for the recovery of nucleic acids from a liquid medium wherein magnetic beads to which the nucleic acid molecules become bound or are adsorbed thereon, on the basis of nonspecific bonds are added to this medium. However, in all three documents a cell lysis step is described before addition of the magnetic beads, which is an obstacle to cell recovery or purification by means of the beads, or else cell recovery by centrifugation before the purification of the nucleic acids, only the latter being effected by means of the magnetic beads. Adsorption of intact cells onto the magnetic beads is not mentioned in the state of the art.

The objective purpose of the present invention was to provide a simple, gentle and effective process for cell recovery, cell purification and/or transfection of cells, which moreover enables isolation of polynucleic acids from these cells with no centrifugation steps. This problem is solved by a process for cell purification and/or cell recovery and/or transfection of cells which includes the addition of magnetic beads to intact cells, wherein the magnetic beads are coated with a glass or polymer coating and bear on their surface chemical groups which allows nonspecific binding of the cells to the surface.

Beads which are suitable for the present process can include every type of previously known magnetic beads wherein a magnetic core is coated with a glass or polymer coating, and which bear on their surface groups which enable nonspecific adsorption or binding of intact cells onto the beads. Preferably, beads can be used which bear on their surface acid groups, preferably carboxylic acid groups, phosphoric acid groups or sulphuric acid groups or salts thereof, particularly preferably carboxylic acid groups or salts thereof, where the said groups can be bound directly to the surface or be part of the polymer forming the surface coating, can be bound to the surface via spacer molecules, or can be parts of a compound which are bound to the surface of the beads. In a preferred embodiment, the beads bear on their surface an overall weak negative total charge, since the cells are particularly effectively bound in the presence of such conditions. A neutral to weakly positive charge on the beads is also possible, even though not preferable for the present process. Examples of suitable carboxylated polymers which are suitable as coating material for the beads and provide a surface suitable for the invention are described in detail in the currently pending German patent application DE 10 2005 040 259.3 (Qiagen, application date 24 Aug. 2005). Examples of compounds which can be bound to the surface are glycine, hydrazine, aspartic acid, 6-aminohexanoic acid, NTA (nitrilotriacetic acid), PEI (polyethylenimine), polyacrylic acid (PAA), HCl, glycerine, diglyme (diethylene glycol dimethyl ether formula: (CH₃OCH₂CH₂)₂O, glyme: glycol diethers), pentaerythritol, toluene, polyallylamine, Jeffamine 500 (O,O-bis(2-aminopropyl)polyethylene glycol 500), polyethylenehexamine, polyethylenimine, bis-tris (bis(2-hydroxyethyl)imino-tris(hydroxymethyl)methane), DIPEA (N,N′-diisopropylethylamine), or combinations thereof, without being restricted to these. Also preferred magnetic beads are those described in the currently pending German patent application DE 10 2005 058 979.9 (Qiagen, application date 9 Dec. 2005). Such suitable magnetic beads are obtainable on the market.

The cells which can be recovered from media by the process of the present invention are preferably eukaryotic cells, in particular cells which are often used for transfection experiments, such as for example mammalian cells or insect cells, which can be grown as suspension cultures or adhesion cultures, cells from blood or tissue which can be present in mixed or pure cultures, or have been taken up in liquid media after isolation. Cell isolation from whole blood or blood serum is also possible, but in all the said cases cell recovery or purification from a suspension of the cells in a liquid medium is preferable.

On contact with the magnetic beads, the cells are adsorbed onto the beads via nonspecific interactions with their surfaces. These mechanical or electrostatic interactions are possibilities, without it being intended to restrict the invention through these theoretical considerations. The size of the beads in comparison to the cells is of no particular significance role for cell recovery/purification, however, particularly in the case of subsequent transfection, as described below, it is preferable that the beads be smaller than the cells to be adsorbed. Particularly preferably, the beads are at most only ca. half as large as the cross-section of the cells to be recovered and most preferably only at most a quarter as large in cross-section.

Preferably the process is performed under conditions which make it possible for the cells in the intact state to be adsorbed onto the beads, or to bind thereto. Such conditions are present when the interaction of the cells with the surface of the beads is not disturbed, preferably when the cells are present as a suspension in a suitable medium, preferably an aqueous medium, such as for example cell culture medium, buffer with a salt concentration suitable for the cells in question, or another aqueous solution which has no adverse effect on the cells. In addition, the solution/suspending medium should preferably contain no polar compounds in a quantity which disturbs the interaction of the cells with the surfaces of the magnetic beads used. Particularly preferably, the cells are present in culture media suitable for the cells in question, or in aqueous buffer solutions which have a salt concentration not detrimental to the cells in question. These media and buffers are well known to persons skilled in the art in this field and can be found in the normal technical literature. Examples of suitable media are DMEM, RPMI (each with or without FCS and other additives), and PBS as the washing buffer; however, all usual cell culture media for mammalian or insect cells can be used, and all buffers of low to moderate salt content.

The cells are brought into contact with the magnetic beads for a sufficiently long time, i.e. over a period which is sufficient to allow the cells to bind/be adsorbed onto the beads. Such a period should be at least 30 secs, preferably at least 1 min, more preferably at least 3 mins and can be of any length, e.g. overnight, or up to 12 hrs., preferably up to 5 hrs., particularly preferably up to 30 mins.

After the adsorption/bonding of the cells onto the magnetic particles, the cells can be collected or separated from the medium surrounding the cells by application of a magnetic field to the vessel in which the cells are present with the beads. In a preferred embodiment, a magnet is placed outside against the vessel in which the cells and the magnetic beads are situated, and the remaining suspending medium is poured out of the vessel or removed by means of a suitable device, for example aspirated with a pipette. The cells can be resuspended in a suitable washing medium and washed with this, by again applying a magnetic field to the vessel, and again removing the washing medium from the vessel. As a result, the present process provides particularly gentle handling of the cells, which is beneficial for the quality of the cells for a subsequent treatment, e.g. in the form of a transfection.

In a preferred embodiment, the recovered and optionally purified cells are next subjected to a transfection, wherein the cells need not be removed from the magnetic beads, but rather in a particularly preferred embodiment remain bound/adsorbed on the beads. The advantage of this adsorption onto the magnetic beads is firstly that no further processing step is necessary for cells before a transfection is performed, and secondly that the transfection rate of suspended cells after adsorption onto the beads to be used according to this invention is increased. One theory, to which it is not intended that the invention be bound, as to why such an improvement in the transfection rate can be achieved is that cells occurring free in suspensions individually each only present a small surface to the transfection compounds, whereas the cells as a whole in the state where they are adsorbed onto the beads offer a larger area, since they are present in the form of a “cross-linked” structure, in which the cells are adsorbed on top of one another via the beads lying between them. The transfection system used can remain attached (lying) on such a structure more easily, so that overall the achievable transfection rate increases.

Preferred transfection systems for the transfection of eukaryotic cells are those in which transfection reagents are added to the cells, and are then taken into the cells via endocytosis. These reagents can for example contain cationic lipids, dendrimers, polyethylenimines, modified polyethylenimines and mixtures thereof. These are adsorbed onto nucleic acids (DNA or RNA in any form used for and suitable for transfection) and thus form “transfection complexes”. These attach to the cells and weakly adhere to the surface until they are taken up via endocytosis. Such transfection reagents are obtainable on the market from various suppliers. Examples of preferred transfection reagents are PolyFect®, Effectene®, Superfect® (all for plasmid transfection; Qiagen, Germany); TransMessenger™ (RNA and siRNA transfection; Qiagen, Germany); RNAiFect™ or HiPerFect (siRNA transfection; Qiagen, Germany).

The transfection efficiency can also in particular be increased in cells which can only be isolated in small quantity, e.g. from blood or tissue, and can be “presented” to the transfection complexes in a markedly more concentrated way in the form of adsorbed cells on the magnetic beads.

After the transfection, the cells can be directly further cultured under growth conditions and in media suitable for the cells. Removal of the magnetic particles is not necessary.

The cells bound to the magnetic beads can be subjected to a step for cell lysis. Here the cells can be lysed either directly after the cell recovery step, or after the purification step or optionally after the transfection step (also after a certain period of culturing). The lysis of the cells takes place after addition of a suitable lysis buffer, or by the application of suitable lysis conditions, and here any condition and any procedure which results in cell opening and hence to the lysis of the cells is suitable. Preferably, lysing solutions are added to the cells, in particular solutions of proteinase K and/or buffers of suitable salt concentrations, or the cells are taken up in deionised water, which is optionally treated with additional lysing components. Suitable lysing conditions for various cell types are well known to persons skilled in the art in this field, and can be found in the literature.

The lysis conditions themselves are not a limiting feature for the present invention, however lysis conditions which result in a solution from which the polynucleic acids liberated by lysis can bind directly to the magnetic beads, such as for example lysis at high salt concentrations in the lysis buffer are preferable. Thus in a preferred process according to the present invention, the same magnetic beads, onto which the intact cells were bound/adsorbed before the lysis can serve directly after the lysis for binding the polynucleic acid molecules liberated from the cells. By addition of a suitable buffer system or by application of suitable conditions, the liberated polynucleotides are adsorbed onto the magnetic beads present in the suspension. The conditions are preferably selected such that the adsorption of the nucleic acids is preferred to the adsorption of the other cell fragments after the lysis.

The nucleic acid molecules bound to the magnetic beads can be separated from the lysis mixture by once again applying a magnetic field to the vessel in which the beads/the nucleic acid molecules are located.

Preferred magnetic beads for use in at least one, preferably at least two, particularly preferably all three component steps according to the invention which are described above, are those which bear the following groups on their surface: glycine, 6-amino-hexanoic acid, aspartic acid, NTA, PEI/PAA, PEI/PAA/NTA, HCl, glycerine/toluene, glycerine/diglyme, pentaerythritol/diglyme, hydrazine, aspartic acid/hydrazine, bis-tris/diglyme or DIPEA/diglyme or combinations of the aforesaid. Also suitable are NTA/NiSO₄, PEI/PAA/NTA/NiSO₄, 6-aminohexanoic acid/NTA/NiSO₄, PEI/toluene/NTA/NiSO₄, PEI/diglyme/NTA/NiSO₄, polyethylenimine, Jeffamine 500 or bis-tris/toluene.

The present process can be performed by the use of mutually matched components and buffer solutions, which can be provided in the form of a kit. For example, such a kit can contain magnetic beads which can be adapted for the cell recovery, cell purification and/or transfection of certain cells, an additive which can bring about the lysis of the cells and optionally a purification buffer for the isolation of the polynucleic acid. In a preferred embodiment, such a kit contains magnetic beads which have a weakly negative overall charge on their surface, for example due to binding of carboxylate groups, a lysis buffer and a purification buffer for nucleic acid molecules.

An advantage of the process steps according to the invention is that removal or changing of the beads used is not necessary during or after any of the steps of the process, but rather the beads used can be the same in all the stated steps, i.e. cell recovery, cell purification, transfection, lysis and nucleic acid purification (even if they are performed independently of one another). Here it is also not necessary for all the steps to be performed with use of the beads, for example the cell recovery and/or purification can also be effected without beads and the beads be used only from the transfection step, however their use from the cell recovery up to transfection and optionally lysis and nucleic acid purification is preferable. A further advantage of the process steps according to the invention is that in each case no centrifugation steps are necessary, as a result of which each of the individual steps can be completely automated. Also the course of the whole process from the cell recovery up to RNA isolation and detection can be fully automated, since the beads do not have to be removed from the preparations at any time, and hence there is no transfer of the vessels which contain the cells/beads into an external device (e.g. a centrifuge). Thus the whole process can be performed in a single device, which has at least one facility for addition of reagents (e.g. a pipetting device) and at least one device for the application of a magnetic field to the vessel which contains the beads. Hence such a device for the automatic implementation of the process according to the invention is also a subject of the present invention.

Figures:

FIG. 1 shows the results of the cytotoxicity test according to Example 2 with HeLa cells.

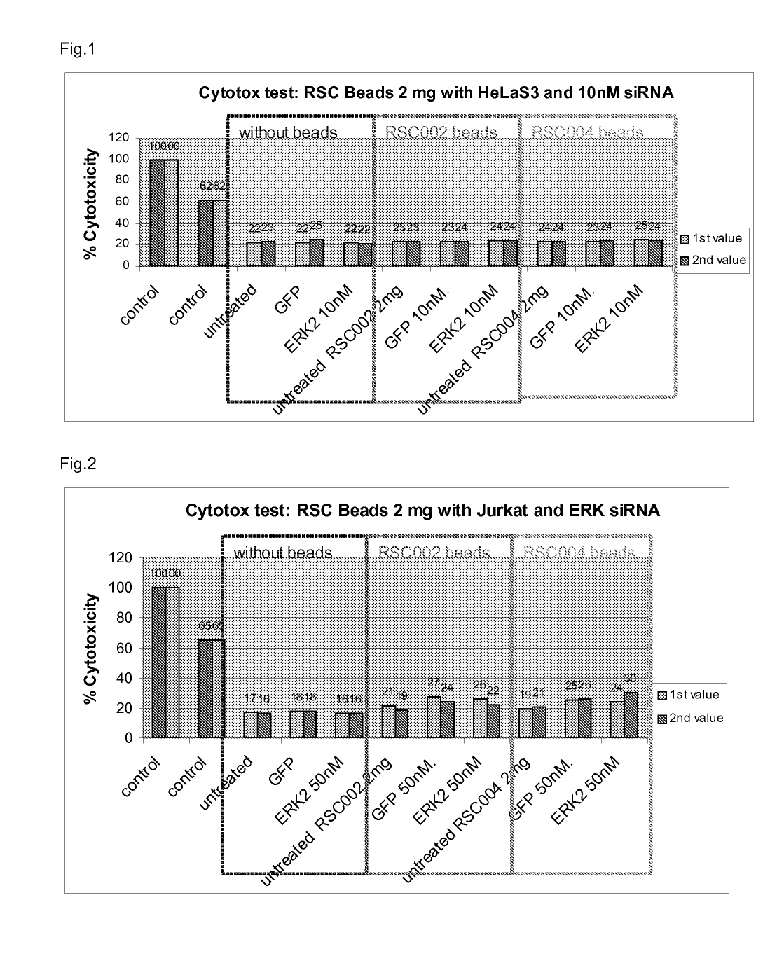

FIG. 2 shows the results of the cytotoxicity test according to Example 2 with Jurkat cells.

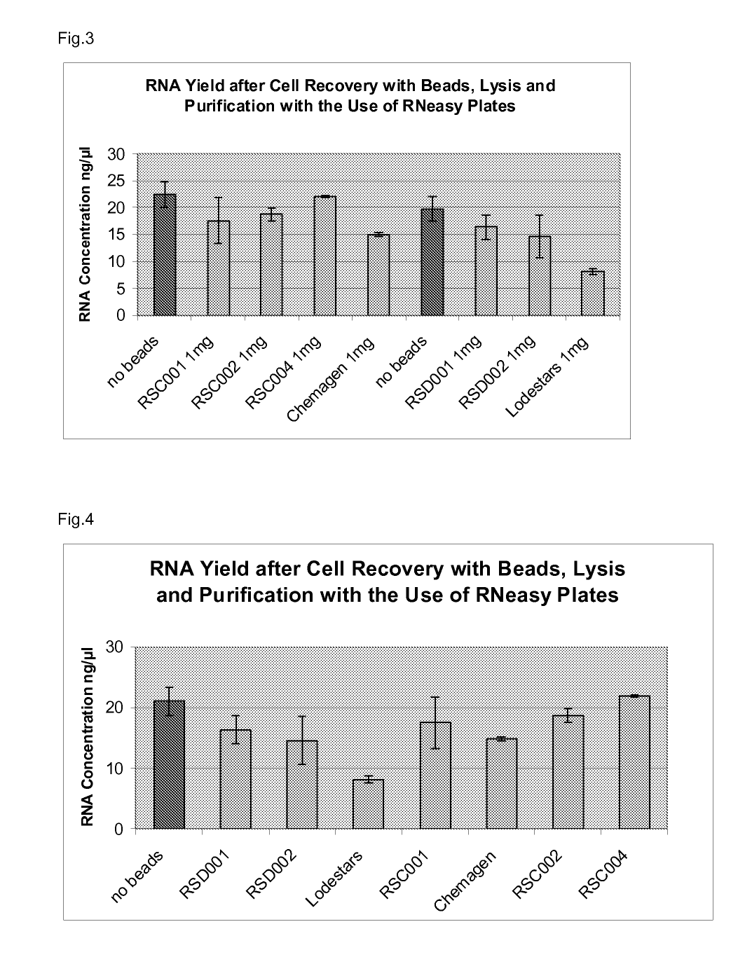

FIGS. 3 and 4 show the yield of RNA which can be isolated from cells bound to beads according to Example 3.

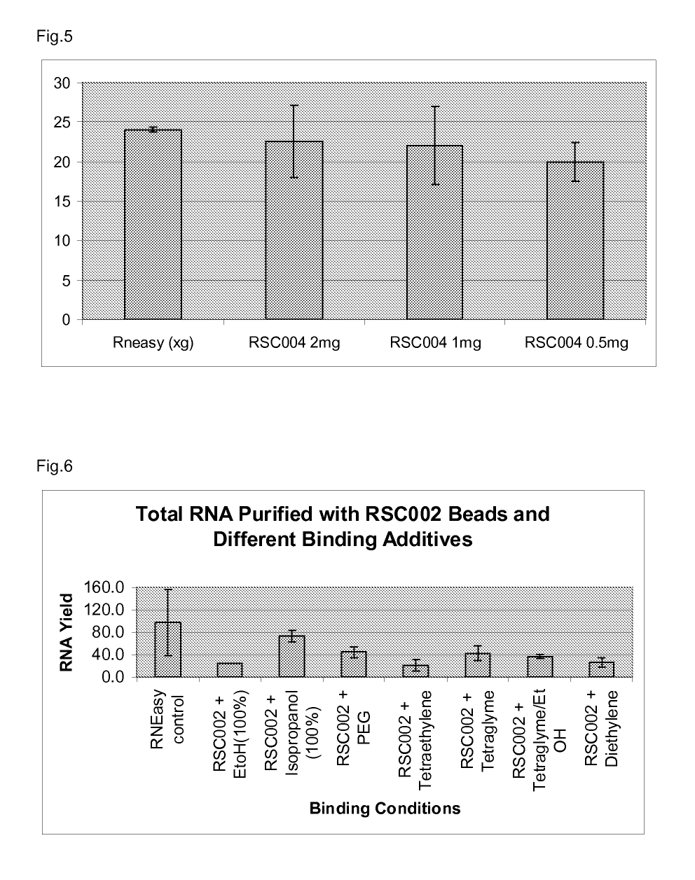

FIG. 5 shows the quantification of the RNA yield from cells in relation to the bead concentration used.

FIG. 6 shows the results of RNA isolation with the use of binding additives according to Example 4

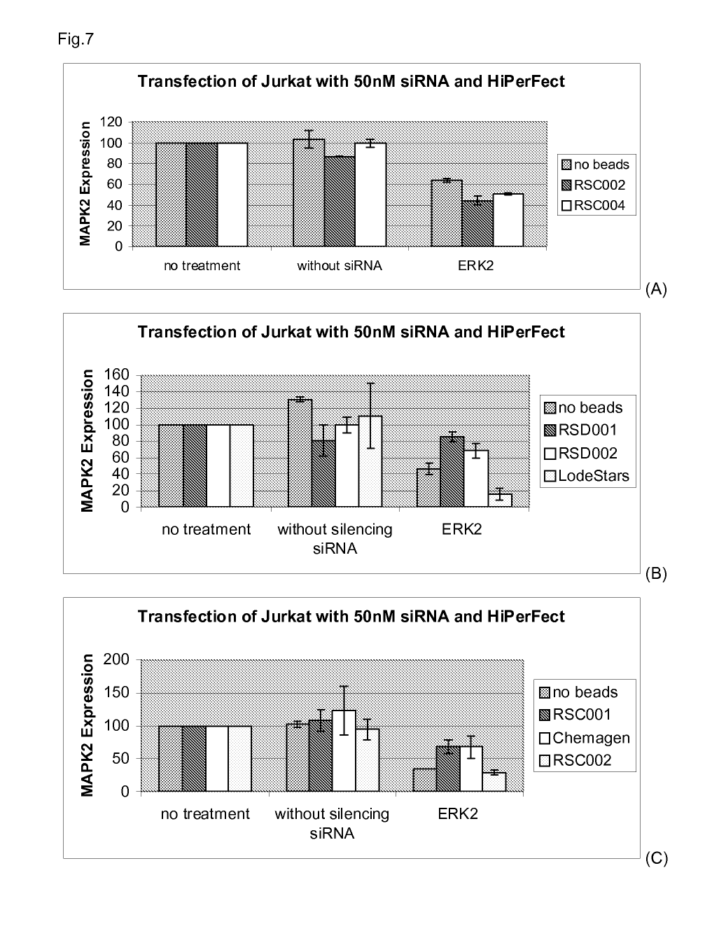

FIGS. 7 (A) (B) and (C) show the efficiency of transfection of cells with the use of beads according to Example 5

The following examples serve only for illustration of the invention and these are not intended in any way to restrict this to the embodiments shown therein.

### EXAMPLES

### Example 1

### Cell Binding of Magnetic Beads with Different Surface Modifications

To a culture of eukaryotic cells (HeLaS3, Jurkat) with a cell density of HelaS3 6×10⁴, Jurkat 1.2×10⁵/500 ml was added a defined quantity of magnetic beads which corresponds to a cells-to-beads ratio of 1 mg/500 μl cell suspension. After incubation for 10 mins, the binding of the cells onto the beads was observed under the microscope, before and after a magnetic field was applied onto the wall of the vessel in which the cell/bead mixture was located. Table 1 shows the binding of the cells onto the beads.

TABLE 1

| Name | Modification | Cell binding |
|---|---|---|
| RSC 001 | glycine | ++ |
| RSC 002 | 6-aminohexanoic acid | ++ |
| RSC 003 | aspartic acid | ++ |
| RSC 004 | NTA | ++ |
| RSC 005 | PEI/PAA | ++ |
| RSC 006 | PEI/PAA/NTA | ++ |
| RSD 002 | HCl | ++ |
| RSD 003 | glycerine/toluene | ++ |
| RSD 004 | glycerine/diglyme | ++ |
| RSD 005 | pentaerythritol/toluene | ++ |
| RSD 006 | pentaerythritol/diglyme | ++ |
| RSE 006 | Jeffamine 500 | + |
| RSE 007 | polyallylamine (20%) | +/− |
| RSE 008 | pentaethylenehexamine | +/− |
| RSE 012 | polyethylenimine linear | + |
| RSE 014 | bis-tris, toluene | + |
| RSE 015 | bis-tris, diglyme | ++ |
| RSE 017 | DIPEA, diglyme | ++ |
| RSH 001 | hydrazine | ++ |
| RSH 002 | aspartic acid/hydrazine | ++ |
| RSH 003 | PEI/PAA/NTA | ++ |
| RSH 004 | PEI/PAA/NTA | ++ |
| RSN 002 | NTA/NiSO4 | + |
| RSN 003 | PEI/PAA/NTA/NiSO4 | + |
| RSN 005 | PEI/PAA/NTA/NiSO4 | + |
| RSN 006 | 6-aminohex. acid/NTA/NiSO4 | + |
| RSN 007 | PEI/PAA/NTA/NiSO4 | + |
| RSN 008 | PEI/PAA/NTA/NiSO4 | + |
| RSN 010 | PEI/toluene/NTA/NiSO4 | + |
| RSN 011 | PEI/diglyme/NTA/NiSO4 | + |

++ very good cell binding, with no significant content of free cells
+ good binding with few free cells
+/− moderate binding with significant content of free cells

### Example 2

### Measurement of the Cytotoxicity of Magnetic Beads in Cell Cultures

The toxicity of the beads is determined by adding a quantity of 0.004 g/ml of the magnetic beads to a cell culture during incubation in the growth phase of the cells. The cells are incubated under suitable growth conditions in a suitable medium for 24 hrs and then examined for cytotoxicity with the Roche LDH cytotoxicity detection kit according to the manufacturer's instructions (Roche). For this, the culture supernatants are collected before the cell lysis and the LDH (lactate dehydrogenase) activity measured. The quantity of LDH released into the medium from the cytosol of destroyed cells is a measure of the cytotoxicity of foreign substances present in the culture.

Growth conditions: HeLaS3 cells were incubated in DMEM (Dulbecco's modified Eagle Medium), complemented with 10% FCS (foetal calf serum) at 37° C. and 5% CO₂ in air. Jurkat cells were incubated in RPMI (Roswell Park Memorial Institute Medium), complemented with 10% FCS (foetal calf serum) at 37° C. and 5% CO₂ in air. The results of the cytotoxicity tests are reproduced in FIGS. 1 and 2.

### Example 3

### Quantification of the Cell Binding Capacity of the Magnetic Beads

The cell binding capacity of the beads is determined by dividing a fresh cell culture of suspended cells into aliquots of equal volume, adding 1 mg of the magnetic beads to each aliquot of the cell culture, applying a magnetic field after 10 mins incubation time and quantifying the cell binding capacity of the beads by total RNA isolation from the bound cells. In order to perform the RNA isolation independently of the type of beads used, the isolation is performed by means of RNEasy 96® plates (Qiagen) and the corresponding isolation protocol. As a control for the cell quantity which can be obtained, the cells from one of the aliquots are recovered by centrifugation and the RNA isolated under the same conditions as for the other samples. The RNA isolated is determined by spectrometry (Spectramax). The results for the RNA concentrations obtained are reproduced in FIGS. 3, 4 and 5. The designation “without beads” represents the purification from the control preparation, in which the cells are recovered by centrifugation. The quantification via the RNA isolated displays a relationship to the cell binding capacity of the individual beads studied per mg beads (FIGS. 3 and 4). In FIG. 5, it can be seen that a further increase in the quantity of the beads used beyond a certain ratio of beads to cells does not increase the yield further.

### Example 4

### RNA Isolation with the Use of Binding Additives

4×10⁵ K562 cells were lysed with 350 μl of RLT buffer and mixed with 7.5 mg of RSC002 beads. Concerning Magattract® A and G beads it is known that a larger quantity of beads is necessary for the total RNA purification than were used in the previous experiments for the binding and transfection of the cells. In order to improve the binding to the beads, screening was performed for binding additives which were known from previous studies to reinforce the binding to beads. A selection of such binding additives is shown in Table 2.

In each case, 350 μl of binding additive were added to the cell lysate with the beads, and the lysate was mixed well. Next, the beads/RNA were washed 2× with 500 μl of RWI buffer and 2× with 600 μl of RPE buffer (buffer from the RNEasy handbook). Next the beads were dried for 10 mins at room temperature and the total RNA was eluted with 70 μl of RNAse-free water. The total RNA was then quantified by spectrometry. The yield of the RNA obtained can be seen from the following Table 3 and is shown in FIG. 6.

TABLE 2
K562 cells and RSC002 beads

| | |
|---|---|
| 1 | Control: cells that had been recovered by centrifugation + RLT + |
| | EtOH (70%) |
| 2 | Cells + RSC002 + RLT + EtOH(100%) |
| 3 | Cells + RSC002 + RLT + isopropanol (100%) |
| 4 | Cells + RSC002 + RLT + PEG 800 |
| 5 | Cells + RSC002 + tetraethylene (tetraethylene glycol) |
| 6 | Cells + RSC002 + RLT + tetraglyme (tetraethylene glycol dimethyl |
| | ether) |
| 7 | Cells + RSC002 + RLT + tetraglyme/EtOH (73.5% tetraethylene |
| | glycol dimethyl ether (257 μl) and 24% EtOH (84 μl)) |
| 8 | Cells + RSC002 + RLT + diethylene (diethylene glycol monoethyl |
| | ether acetate) |

TABLE 3

| Nr | Sample ID | Mean | SD |
|---|---|---|---|
| 1 | RNEasy control | 96.7 | 58.8 |
| 2 | RSC002 + EtOH(100%) | 23.7 | 0.1 |
| 3 | RSC002 + isopropanol | 72.8 | 10.9 |
| | (100%) | | |
| 4 | RSC002 + PEG | 45.3 | 9.5 |
| 5 | RSC002 + tetraethylene | 21.3 | 10.7 |
| 6 | RSC002 + tetraglyme | 42.5 | 13.5 |
| 7 | RSC002 + tetraglyme/EtOH | 36.6 | 3.1 |
| 8 | RSC002 + diethylene | 26.6 | 8.9 |

→This screening shows that the binding conditions can be very well optimised and that the beads are also suitable for RNA purification.

### Example 5

### Transfection Efficiency

The effect of the beads on the transfection efficiency was tested by transfection experiments with Jurkat and HeLAS3 cells using the HiPerFect transfection reagent according to the manufacturer's instructions (Qiagen), using/transfecting 50 nM MAPK2 siRNA (silencing RNA, designated as “ERK2”) each time. As controls, on the one hand cells were transfected in the absence of beads, and on the other hand the transfection process was performed in the presence of the beads with no siRNA. As can be seen from FIGS. 7 (A), (B) and (C), the presence of the beads during the transfection assists the uptake and increases the transfection efficiency, which manifests itself in a decrease in the MAPK2 expression due to “silencing” of this gene. “No treatment” stands for the MAPK2 expression of completely untreated cells.

1. Process for the transfection of cells which includes the addition of magnetic beads to intact cells, wherein the magnetic beads include a glass or polymer coating and bear on their surface chemical groups which allows nonspecific binding of the cells to the surface.

2. Process according to claim 1, wherein before the transfection, a cell purification and/or cell recovery by means of the same beads is performed.

3. Process according to claim 1, wherein the beads are added to a cell suspension.

4. Process according to claim 1, wherein the method is performed under conditions which make it possible for the cells to be adsorbed onto the beads or to bind thereto in an intact state.

5. Process according to claim 1, wherein after the adsorption/binding of the cells onto the beads, a transfection of the adsorbed/bound cells with polynucleotide molecules is effected.

6. Process for the isolation of polynucleotides from cellular material including a process according to claim 1, wherein after the adsorption/binding of the cells onto the beads and optionally after the transfection, a lysis of the adsorbed/bound cells is effected.

7. Process according to claim 6, wherein after the lysis of the cells, a separation of the polynucleotides from other cell components is effected.

8. Process according to claim 7, wherein the separation of the polynucleotides from the other cellular components is effected by the poly-nucleotides remaining adsorbed/bound onto the magnetic beads while the other cellular components essentially do not bind onto the beads.

9. Process according to claim 8, wherein the separation is effected by means of a magnet.

10. Use of magnetic beads which are coated with a glass or polymer coating and bear on their surface chemical groups which allows nonspecific binding of the cells to the surface for the transfection of cells.

11. Kit for the implementation of a process according to claim 1, comprising magnetic beads which are coated with a glass or polymer coating and bear on their surface chemical groups which allows nonspecific binding of the cells to the surface.

12. Device for the implementation of the process according to claim 1, comprising at least one addition device and at least one device for the application of a magnetic field to a vessel wall.
